(19) United States
(12) Patent Application Publication
(10) Pub. No.: US 2023/0400464 A1
(43) Pub. Date: Dec. 14, 2023

(54) METHOD FOR ASSESSING THE SEVERITY OF SARS

(71) Applicant: ABIONIC SA, Epalinges (CH)

(72) Inventors: Nicolas DURAND, Blonay (CH); Iwan MÄRKI, Yverson-les-Bains (CH); François VENTURA, La Croix sur Lutry (CH)

(21) Appl. No.: 18/251,321

(22) PCT Filed: Nov. 3, 2020
(86) PCT No.: PCT/IB2020/060314

§ 371 Date: May 1, 2023

(51) Int. Cl. G01N 33/569; G01N 33/68

(52) U.S. Cl. CPC G01N 33/56983; G01N 33/6893; G01N 2333/165

(57) ABSTRACT

The present invention relates to a method for assessing the severity of Severe Acute Respiratory Syndrome (SARS) in a patient; said method being based on a severity score consisting of at least: a) a respiratory system score, b) a score determined by the level of pancreatic stone protein/regenerating protein (PSP/reg) in a body fluid sample from said patient. The invention may be used in a healthcare unit for sorting patients to decide the treatment priority

### FIELD OF INVENTION

The present invention relates to a method for assessing the severity of Severe Acute Respiratory Syndrome (SARS), in particular SARS-CoV-2 (Covid-19). Such a method may be used in a healthcare unit for sorting patients to decide the treatment priority.

### Definitions

As defined in the present document, patients infected with SARS-CoV-2 refers to patients that have been positively tested with any method identifying the presence of SARS-CoV-2.

“PSP/reg” refers to human pancreatic stone protein, also called regenerating gene (REG) I protein or lithostatine or pancreatic thread protein (Gross et al., J. Clin. Invest. 1985, 76:21 15-2126) and can be the isoform alpha (Uniprot sequence number: P05451, also identified herewith as SEQ ID NO:1) or beta (Uniprot sequence number: P48304, also identified herewith as SEQ ID NO:2).

### BACKGROUND OF THE INVENTION

The rapid and global spread of the SARS-CoV-2 and the associated Covid-19 pandemic poses multiple challenges to public health systems. This is particularly true for intensive care units (ICU) where the rapidly increasing number of patients requiring invasive respiratory support places a severe burden on ICU healthcare professionals, infrastructures, material and treatments needs. The impressive amount of data published over the past few months has shed light on the clinical characteristics of Covid-19 patients, including risk factors for severe disease and death. Various studies of Covid-19 ICU patients have shown differences in mortality rates, but have consistently reported high numbers (F. Zhou, and al., The Lancet 2020), ranging from 61-97%. Patients infected by SARS-CoV-2 that are generally defined as “at risk” (older age and/or patients with comorbidities such as cardiovascular disease, diabetes, chronic respiratory disease, hypertension, and cancer (R. E. Jordan, et al., BMJ 2020)) have a higher likelihood of experiencing complications of Covid-19, mostly severe pneumonia, septic shock and multiple organ dysfunction. In a recent study, 11% of Covid-19 patients who developed acute respiratory distress syndrome (ARDS) worsened over a short period of time and later died of multiple organ failure (N. Chen, et al., The Lancet 2020, 10223:507-513).

Sepsis is currently defined as “a life-threatening organ dysfunction caused by a dysregulated host response to infection” (Sepsis-3) (M. Singer, and al., JAMA 2016, 315:801-810). Clinically, organ dysfunction is defined as sequential organ failure assessment (SOFA) score of two points or more. A subset of septic patients progresses to septic shock. These septic shock patients are clinically defined as having persistent hypotension requiring vasopressors to maintain a mean arterial pressure of >65 mm Hg and with a serum lactate level greater than 2 mmol/L (18 mg/dL) despite adequate volume resuscitation. From these definitions it is apparent that all SARS-CoV-2 infected patients who required invasive mechanical ventilation due to lung failure shall be defined as “septic”, because of the SOFA respiratory points≥2.

In this context, it is important to identify early onset identifiers of Covid-19 complications such as septic shock and multiple organ dysfunction. The SOFA score, apart from being cumbersome to measure due to the multiple parameters comprising it and the need of a central laboratory to collect all data, is not predictive of organ dysfunction but rather an expression of the extent of organ dysfunction at a given time. Therefore, there is a need for early alert signal(s) of organ dysfunction and septic shock to provide clinicians with timely data for patient management to mitigate the impact of these dysfunctions and promptly reset homeostasis. Septic shock and multiorgan dysfunction are major complications in critically ill Covid-19 patients admitted to intensive care units (ICU) and are associated with high mortality rate. Biomarkers that are predictive of these conditions are therefore essential for early diagnosis in order to best adapt the patient management strategy.

Nowadays the diagnosis can only be done by the combination of multiple clinical signs, the complex sequential organ failure assessment score (SOFA score) and the controversial Sepsis-3 definition (S. Sinha et al., J Anaesthesiol Clin Pharmacol. 2018, 34:4:542-543), and not by a simple blood test. Other biomarkers, Procalcitonin PCT and C-reactive protein CRP, have extensively been studied, but today none of them have shown the capacity to detect sepsis quickly enough and with a high enough diagnostic accuracy (C. Rhee, et al., Critical care 2016, 6:20:89).

Pancreatic stone protein/regenerating protein (PSP/reg) belongs to a family of lectin-binding proteins that allows a much earlier diagnosis of sepsis, septic shock and organ dysfunction than all other current solutions proposed in the international guidelines (C. S. Singer, et al. JAMA 2016, 315:8:801-810) of medical societies.

A literature review (P. Eggimann, et al., Biomarkers 2019, 13:02:135-145), summarizing 13 PSP/reg studies, was published in 2019 and a promising study (H. J. Klein, et al. Ann Surg. 2020) was published in January 2020 by the Burn center of the University Hospital of Zurich.

Theses numerous publications show that the PSP/reg assay is more specific and sensitive than other biomarker for the early diagnosis of sepsis, septic shock and multiorgan failure in ICU adults (M. Llewlyn, et al., Crit. Care 2013, 17:2:R60), children (Z. Jiri, et. al., Cytokine 2014, 66:2:106-111), newborn (A. Rass, et al., BioMed. Res. Int 2016, 1-8) and burn (H. J. Klein, et al., World Journal of Surgery 2020, 44:3000-3009) patients. PSP/reg allows the identification of sepsis in post-cardiac surgery patients (H. Klein, et al., PLoS ONE 2015, 10:3:e0120276), PSP/reg predicts outcome in patients with peritonitis in ICU (R. Gukasjan, et al., Crit. Care Med 2013, 41:4:1027-1036), PSP/reg is a biomarker of organ failure in Ventilator-Associated-Pneumonia VAP (L. Boeck, et al., Chest 2011, 140:4:925-932) and a biomarker predicting mortality in adults (Y.-A. Que, et al., Crit. Care 2012, 16:4: R114) and in children (Q. Wu, et al., Med. Sci. Monit 2017, 23:1533-1539).

The values of PSP/reg were determined in the healthy subject in 2015 (E. Schlapbach, et al., BMC Anesthesiol 2015, 15:168). PSP/reg detect early infection and sepsis in traumatized patients (M. Keel, et al., Crit. Care Med 2009, 37:5:1642-1648), in emergency department (L. Garcia de Guardinia-Romualdo, et al., Eur. J. Clin. Invest 2017, 47:4:297-304), in pediatric acute osteomyelitis (C. Cui, et al., Med. Sci. Monit 2017, 23:5211-5217), and in cancer patients with febrile neutropenia (L. Garcia de Guadiana-Romualdo, et al., Clin Chem Lab Med 2019, 57:4:540-548).

The pathophysiological mechanism of PSP/reg is not yet clearly defined (R. Graf, Pancreatology 2020, 20:3:301-304), but studies in rats suggest that that serum PSP/reg in septic patients is predominantly derived through an acute phase response of the pancreas (T. Reding, et al., Oncotarget 2017, 8:30162-30174). In humans, PSP/REG activates granulocyte neutrophils (M. Keel, et al., Crit. Care Med 2009, 37:51642-1648), which seems to confirm that the PSP/reg might serve as an acute phase protein.

The association between changes in biomarker concentrations in the development of sepsis (bacterial and viral), septic shock and multiorgan dysfunction was assessed in this invention to identify a new diagnostic strategy for early identification of complications in Covid-19 patients.

Currently, it is very difficult to identify clinical deteriorations of hospitalized SARS patients, in particular Covid-19 patients, as most of them have sepsis due to the viral infection, and to the respiratory distress. Moreover, the cytokine storm is creating a complex clinical picture making it difficult for the practitioners to assess the severity of the clinical situation.

A fortiori, there is presently no score to help the clinicians to sort and/or orient patients for better treatment.

There is therefore a strong need for solutions to speed up and improve the assessment of the severity of SARS.

### GENERAL DESCRIPTION OF THE INVENTION

The inventors have discovered that the severity assessment of SARS, especially SARS-CoV-2, may be facilitated and its quality improved with the use of a severity score that is composed of at least:
a. a respiratory system score,
b. a score determined by the level of pancreatic stone protein/regenerating protein (PSP/reg) in a body fluid sample from said patient.

According to a preferred embodiment of the invention, the method comprises the following steps:
a. Providing a body fluid sample from said patient;
b. Determining the level of pancreatic stone protein/regenerating protein (PSP/reg) in said sample;
c. Determining the PSP/reg score;
d. Determining the respiratory score;
e. Determining the severity score by adding the PSP/reg and respiratory score.

The severity score may also advantageously include another biomarker score, such as a C-Reactive Protein (CRP).

The severity score is indicative of the development of clinical complications and/or deteriorations including multiple organ failures, bacterial sepsis and severe viral sepsis.

The PSP/reg score correlates with the sequential organ failure assessment score (SOFA score).

The invention allows triage, sorting, orientation, positioning and/or stratification of patients according the severity level.

### DETAILED DESCRIPTION OF THE INVENTION

The invention is explained in a more precise way in the present chapter, with the support of the following figures:

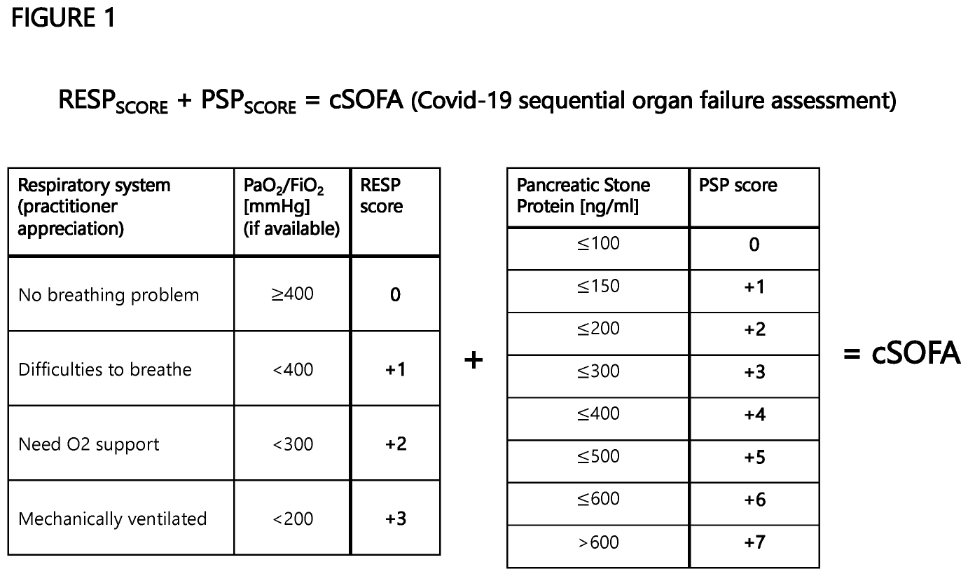

FIG. 1 shows how the severity score of Covid-19 patients is determined. The severity score called Covid-19 sequential organ failure assessment (cSOFA) is composed of the sum of the respiratory score (RESP_{SCORE}) and the PSP score (PSP_{SCORE}). The RESP_{SCORE} is established by the practitioner as indicated in the table and directly linked to the PaO₂/FiO₂ measurement also known as the respiratory dysfunction component of the SOFA score. The PSP_{SCORE} is derived from the measured PSP/reg level as indicated in the table.

RESP_{SCORE}+PSP_{SCORE}=cSOFA (Covid-19 sequential organ failure assessment)

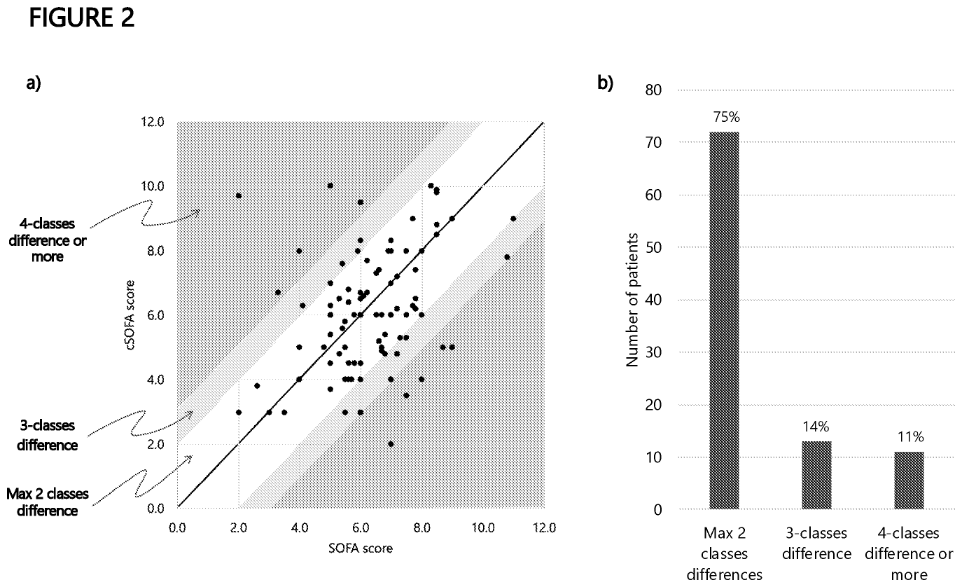

FIG. 2a shows the correlation between the cSOFA score and the generally known SOFA score. Each point represents an averaged cSOFA-SOFA comparison over the ICU stay for each patient. 96 patients at ICU have been included in this analysis. The white area represents a maximum of 2 classes difference between cSOFA and SOFA scores. The light grey area represents a maximum of 3 classes difference between cSOFA and SOFA scores, and the dark grey area represents a 4-classes or more difference between cSOFA and SOFA scores. FIG. 2b illustrates the distribution of patients included in the analysis, showing cSOFA and SOFA scores class differences.

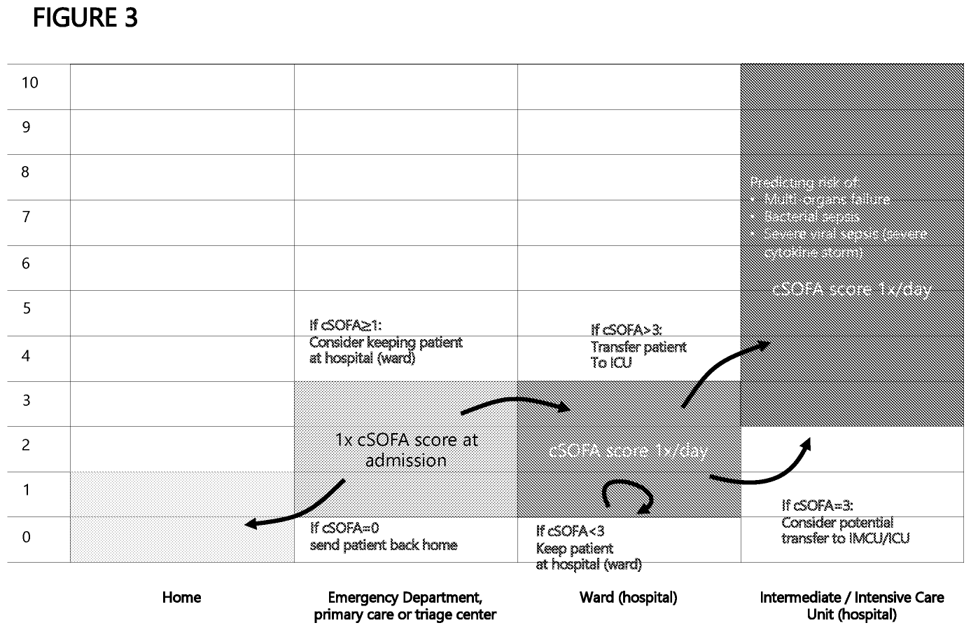

FIG. 3 shows how the cSOFA is interpreted according its level for the positioning of the patient clinical severity and his orientation. A cSOFA score equal to zero indicates a low risk of clinical deterioration, thus the orientation of sending the patient home should be considered. A cSOFA score between 1 and 3 indicates a medium risk of clinical deterioration, thus hospitalization should be considered. A cSOFA score above 3 indicates a high risk of clinical deterioration, thus transferring the patient to intermediate care unit or intensive care unit should be considered.

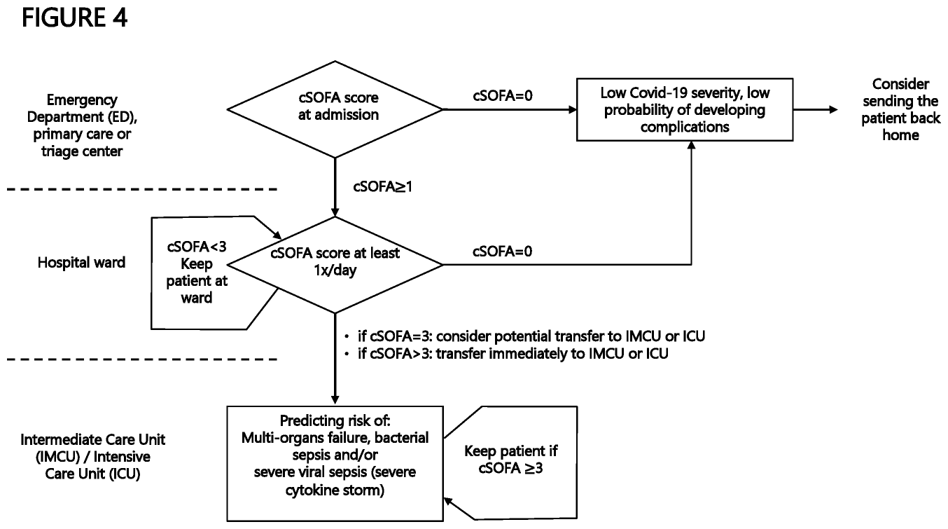

FIG. 4 shows a decision tree for triage and orientation of patients according to the cSOFA score level as illustrated in FIG. 3.

As defined in those illustrated examples that refer to SARS-CoV-2, the severity score is called Covid-19 sequential organ failure assessment (cSOFA) and is composed of the sum of the respiratory score (RESP_{SCORE}) and the PSP score (PSP_{SCORE}). The RESP_{SCORE} is identical to the respiratory dysfunction component of the SOFA score. It may be established by the practitioner's appreciation as shown in FIG. 1 and/or, if available, directly derived from the PaO₂/FiO₂ measurement according the scoring system of the SOFA score. The PSP_{SCORE} is established based on the measured patient level of pancreatic stone protein/regenerating protein PSP/reg in body fluids according to the table set forth in FIG. 1.

Body fluids useful for determination of PSP/reg levels are e.g. whole blood, serum, plasma, urine, sputum, cerebrospinal fluid, tear fluid, saliva, sweat, milk, or extracts from solid tissue or from fecal matter.

Any known method may be used for the determination of the level of PSP/reg in body fluids. Methods considered are e.g. ELISA, RIA, EIA, mass spectrometry, or microarray analysis and any fluidic assay methods.

The obtained cSOFA score from 0 to 10 correlates well to the well-established SOFA score from 0 to 10 for Covid-19 patients as shown in FIG. 2. The SOFA score is an expression of the extent of organ dysfunction at a given time and is widely used today to assess organ failure, sepsis and septic shock. This score is complex and based on multiple clinical parameters and is therefore cumbersome to establish, reason why it is mainly used in intensive care units. The cSOFA score is based on only two parameters that can quickly be measured and calculated. It can be determined easily by any healthcare professional and therefore it is an alternative for the SOFA score. In particular, as the SOFA score is usually used in intensive care settings, the cSOFA score becomes a solution in all other settings (emergency department, general ward, primary care, testing centers, etc.) where the SOFA score is currently not readily available or calculated. In consequence, the use of the cSOFA score in these other settings allows to determine clinical complication, deterioration, triage and orientation of Covid-19 patients.

As described in FIG. 3 a cSOFA score equal to zero indicates a low risk of clinical deterioration, thus the orientation of sending the patient home should be considered. A cSOFA score between 1 and 3 indicates a medium risk of clinical deterioration, thus hospitalization should be considered. A cSOFA score above 3 indicates a high risk of clinical deterioration including risk of multiple organ failures, bacterial sepsis and severe viral sepsis (severe cytokine storm), thus transferring the patient to intermediate care unit or intensive care unit should be considered.

Based on the described classification of the cSOFA score (see FIG. 3) a decision tree for the triage, sorting and orientation of Covid-19 patients is established as shown in FIG. 4. At the emergency department, primary care settings or triage centers the cSOFA score is established once at admission for the purpose of triage and the identification of clinical severity. During hospitalization the cSOFA score is established at least on a daily manner for the same purpose.

The invention is not limited to the assessment of SARS-CoV-2 but may also be advantageously used with patients infected by any type of SARS.

1. A method for assessing the severity of Severe Acute Respiratory Syndrome (SARS) in a patient, said method being based on a severity score composed of at least:
a) a respiratory system score,
b) a score determined by the level of pancreatic stone protein/regenerating protein (PSP/reg) in a body fluid sample from said patient.

2. The method of claim 1 for assessing the severity of SARS-CoV-2 (Covid-19).

3. The method of claim 1 comprising the following steps:
a) Providing a body fluid sample from said patient;
b) Determining the level of pancreatic stone protein/regenerating protein (PSP/reg) in said sample;
c) Determining the PSP/reg score;
d) Determining the respiratory score;
e) Determining the severity score by adding the PSP/reg and respiratory score.

4. The method of claim 1 wherein the severity score also includes another biomarker score.

5. The method of claim 4 wherein said biomarker is C-Reactive Protein (CRP).

6. The method of claim 1 wherein the body fluid sample is a serum, plasma, whole blood sample, saliva, urine, sputum, cerebrospinal fluid, tear fluid, sweat, milk, or extracts from solid tissue or from fecal matter.

7. The method of claim 1 wherein the respiratory score is obtained by assessing how the patient has difficulties to breath, needs oxygen support or mechanical ventilation (invasive and non-invasive).

8. The method of claim 1 wherein a severity score of less than 1 is indicating a low risk of clinical complications and/or deteriorations.

9. The method of claim 1 wherein a severity score greater than 3 is indicative of high risk of developing severe viral sepsis (severe cytokine storm), bacterial sepsis, multiple organ failures and/or death.

10. The method of claim 1 wherein the level of PSP/reg is determined by ELISA, RIA, EIA, mass spectrometry, microarray analysis or any fluidic assay methods.

11. Use of the severity score as defined in claim 1 for patient triage, sorting, orientation, positioning and/or stratification in a healthcare unit.
